(19) United States
(12) Patent Application Publication
(10) Pub. No.: US 2024/0287140 A1
(43) Pub. Date: Aug. 29, 2024

(54) Single Protein-Encapsulated Biopolymers/Bio-oligomers for Therapeutic and Diagnostic Applications

(71) Applicant: Sunstate Biosciences, LLC, Pasadena, CA (US)

(72) Inventor: Changjun Yu, Pasadena, CA (US)

(73) Assignee: Sunstate Biosciences, LLC, Pasadena, CA (US)

(21) Appl. No.: 18/415,225

(22) Filed: Jan. 17, 2024

Related U.S. Application Data
(60) Provisional application No. 63/439,753, filed on Jan. 18, 2023.

(51) Int. Cl. C07K 14/00; C07K 14/765

(52) U.S. Cl. CPC C07K 14/003; C07K 14/765; C07K 2319/60

(57) ABSTRACT

The invention provides compositions comprising a single protein having one or more biopolymers or modified biopolymers for therapeutics and diagnostics tightly bound therein. The compositions are useful to enhance efficacy and water solution of biopolymers, decrease their toxicity and/or to widen therapeutic window of the biopolymers. The invention also provides methods for preparing such compositions.

### CROSS-REFERENCE TO RELATED APPLICATIONS

This application claims priority to U.S. Provisional Patent Application No. 63/439,753, filed on 18 Jan. 2023. The entire content of this United States Provisional Patent Application is hereby incorporated by reference herein.

### BACKGROUND

Biopolymers or Bio-oligomers, such as natural and modified nucleic acids (NA), natural and modified peptides/proteins and various peptide nucleic acids (PNA), and their derivatives, have been used for therapeutical and diagnostical applications in various formats. Many intrinsic problems of those biopolymers or bio-oligomers have limited their applications in the clinical settings. Major hurdles include: (1) short in vivo half-lives, the naturally occurring biopolymers/bio-oligomers (nucleic acids, peptides/proteins) are very susceptible to enzymatic digestion, leading to very short in vivo half-lives, which is unable to reach to the targets; (2) Difficulty crossing cell membranes, due the charged nature, some biopolymers/bio-oligomers, such as nucleic acids, are unable to cross the cell membranes, therefore, the applications can't be achieved; (3) poor water solubility, the backbone-modified NAs (morpholino oligonucleotides), PNA and peptides with unusual sequences have poor water solubility; therefore, those biopolymers/bio-oligomers are very difficult to be druggable.

Various efforts and studies have been undertaken to improve the biopolymers/bio-oligomers for therapeutic and diagnostic applications. The approaches include the nanoparticles, macromolecule micelles and conjugations to lots of the different polymers (naturally and non-natural). The success is very limited. Thus, there is an urgent need for formulations that not only increase their water solubility, but also enhance their efficacies in human clinical settings for anticancer and other indications.

HSA is the most abundant serum protein in the body, with a total of about 460 g distributing among the blood circulation, the lymphatic system and the extracellular/intracellular compartments (Peters, T., 1996, All About Albumin: Biochemistry, Genetics and Medical Applications. San Diego, CA: Academic Press Limited). Its functions include providing essential colloidal osmotic pressure, balancing plasma pH, and binding and transporting hydrophobic molecules such as fatty acids and bilirubin. HSA possesses some unique properties (Hoogenboezem, E. N., and Duvall, C. L., Adv Drug Deliv Rev, 2018, 130, 73-89): 1) being highly soluble and thermally stable, 2) capable of binding a variety of ligands with different binding affinity, 3) being endocytosed and transcytosed into and cross cells via receptors, 4) displaying an unusually long half-life of 19 days due to effective endosome recycling by the neonatal Fc receptor (FcRn) and rescue from renal clearance via Megalin/Cubilin-complexes (Chaudhury, C., J Exp Med, 2003, 197, 315-322; Anderson, C. L., et el., Trends Immunol, 2006, 27, 343-348; Chaudhury, C., et el., Biochemistry, 2006, 45, 4983-4990; and Kim, J., Bronson, et el., Am J Physiol Gastrointest Liver Physiol, 2006, 290, G352-360), 5) able to accumulate at tumor tissues due to EPR effects, and 6) being preferentially taken up and metabolized by cancer cells to serve as nutrients (Stehle, G., et el., Crit Rev Oncol Hematol, 1997, 26, 77-100; Commisso, C., et el., Nature, 2013, 497, 633-637; and Kamphorst, J. J., et el., Cancer Res, 2015, 75, 544-553).

### SUMMARY

Applicant has identified a method to tightly bind “biopolymers” without any net charges, such as PNA (peptide nucleic acids), carbamate oligonucleotides, morpholino oligonucleotides with carbamate linkage, dialkylsilyl oligonucleotides, peptides and small proteins, and “modified biopolymers” which have charged backbones containing variable “hydrophobic moieties”, such as natural and modified nucleic acids, etc., within single proteins (e.g. albumins, antibodies), while substantially maintaining the properties of the single protein. This method provides new compositions that enhance efficacy and water solubility of Biopolymers/Bio-Oligomers as therapeutics and diagnostic proboles and reduce their toxicity as the targeted therapeutics and diagnostics.

In one embodiment, the invention provides a composition comprising a single protein having one or more biopolymer or modified biopolymer tightly bound therein.

The invention also provides a method to treat cancer in an animal comprising administering to the animal a composition that comprises a single protein having one or more biopolymer or modified biopolymer tightly bound therein as anticancer therapeutics.

The invention also provides a method comprising: a) combining a first solution that comprises a biopolymer or modified biopolymer with a second solution that comprises a single protein, water, and a polar organic solvent to provide a third solution; and b) stirring the third solution under conditions that allow one or more biopolymer” or modified biopolymer to become tightly bound within each single protein molecule. The invention also provides a composition prepared by a method of the invention.

The invention also provides a pharmaceutical composition that comprises 1) a single protein with one or more molecules of biopolymer or modified biopolymer tightly bound therein and 2) a pharmaceutically acceptable carrier.

### BRIEF DESCRIPTION OF THE FIGURES

FIG. 1 illustrates an encapsulation process combining a biopolymer and a single protein to form a composition of the invention.

FIG. 2 shows an illustration of the relationship between biopolymers, single proteins, hydrophobic moieties, modified biopolymers, and an encapsulation process for combining a modified biopolymer and a single protein to form a structure including a single protein and a modified biopolymer tightly bound therein.

FIG. 3 illustrates hydrophobic moieties.

FIG. 4 illustrates a biopolymer completely encapsulated by a single protein.

FIG. 5 illustrates a biopolymer wherein only part of the surface area of the biopolymer is encapsulated by a single protein.

FIG. 6 illustrates a biopolymer that is linked to a hydrophobic moiety, where the organic moiety of the hydrophobic moiety is completely encapsulated by a single protein.

FIG. 7 illustrates a biopolymer that is linked to a hydrophobic moiety that comprises an organic moiety, where the organic moiety is partially encapsulated by a single protein (e.g., not completely within the single protein).

FIG. 8 shows the UV Spectra of SPEPNA12605J and HSA in water (Example 1).

FIG. 9 shows the relative cell viability vs concentration of PNA12605J in DMSO solution (Example 1).

FIG. 10 shows the relative cell viability vs concentration of SPEPNA12605J in di water solution (Example 1).

FIG. 11 shows the UV Spectra of SPEPNA118C and HSA in water (Example 2).

FIG. 12 shows the relative cell viability vs concentration of PNA118C in DMSO solution (Example 2).

FIG. 13 shows the relative cell viability vs concentration of SPEPNA118C in DI water (Example 2).

### DETAILED DESCRIPTION

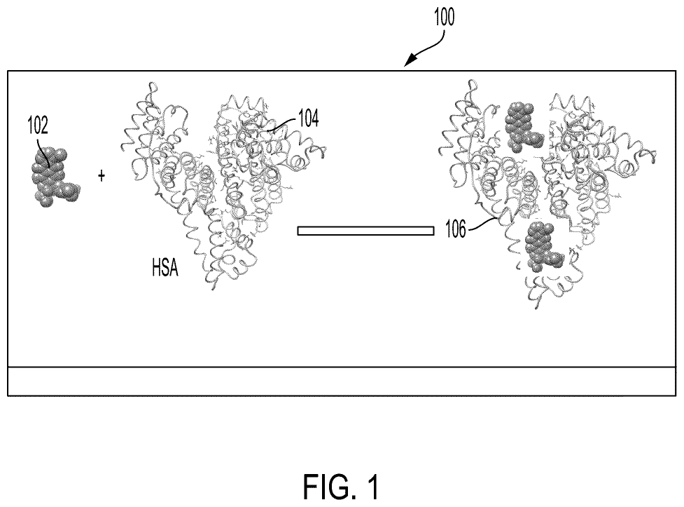

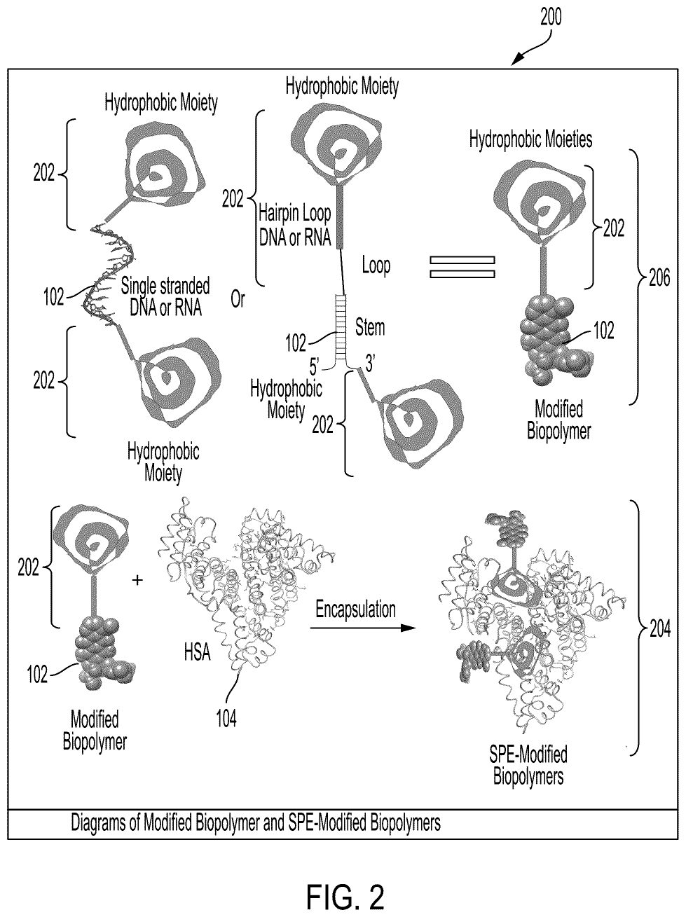

In one embodiment, the invention provides a composition comprising a single protein having one or more units of biopolymer or modified biopolymer tightly bound therein (FIG. 1 and FIG. 2).

Term “biopolymer” (FIG. 1) comprises any natural or modified bioactive polymer that contains no charge and is hydrophobic.

The term “modified biopolymer” (FIG. 2) comprises any natural or modified bioactive polymer that is linked to one or more hydrophobic moieties. In one embodiment, the modified biopolymer is charged and is linked to one or more hydrophobic moieties. In one embodiment, the modified biopolymer is not charged, and it is linked to one or more hydrophobic moieties.

In one embodiment, the invention provides a composition comprising a single protein having one or more biopolymers tightly bound therein, wherein the one or more biopolymers contain no charge and are hydrophobic.

In one embodiment, the invention provides a composition comprising a single protein having one or more modified biopolymers tightly bound therein, wherein the one or more modified biopolymers contain no charge and are each linked to one or more hydrophobic moieties.

In one embodiment, the invention provides a composition comprising a single protein having one or more modified biopolymers tightly bound therein, wherein the one or more modified biopolymers are charged and are each linked to one or more hydrophobic moieties.

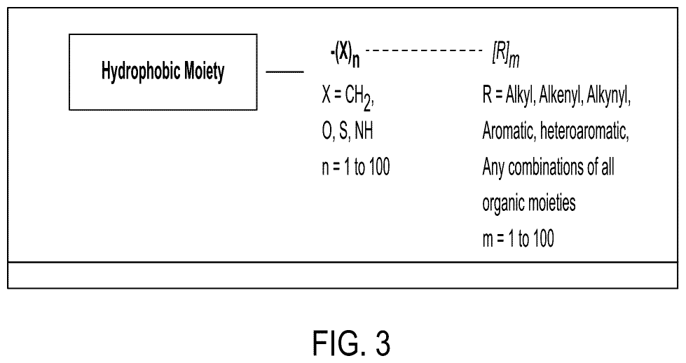

The term “hydrophobic moiety” includes any organic structure or moiety that has low solubility or is insoluble in aqueous media and does not significantly and/or negatively affect the biological properties of the corresponding modified biopolymers. The hydrophobic moiety can comprise various combinations of O, N, S, alkyl, alkenyl, alkynyl, aromatic rings, and heteroaromatic rings with 1 to 100 of those units in any combinations. In one embodiment, the hydrophobic moiety comprises a residue of a fatty acid. In one embodiment, the hydrophobic moiety comprises 8-200 carbons. In one embodiment, the hydrophobic moiety comprises 8-100 carbons. In one embodiment, the hydrophobic moiety comprises 8-50 carbons. In one embodiment, the hydrophobic moiety comprises 15-200 carbons. In one embodiment, the hydrophobic moiety comprises 15-100 carbons. In one embodiment, the hydrophobic moiety comprises 15-50 carbons. In one embodiment, the hydrophobic moiety comprises 8-200 carbons and 1, 2, 3, 4, 5, or 6 atoms independently selected from the group consisting of N, O, and S. In one embodiment, the hydrophobic moiety comprises 8-100 carbons and 1, 2, 3, 4, 5, or 6 atoms independently selected from the group consisting of N, O, and S. In one embodiment, the hydrophobic moiety comprises 8-50 carbons and 1, 2, 3, 4, 5, or 6 atoms independently selected from the group consisting of N, O, and S. In one embodiment, the hydrophobic moiety comprises 15-200 carbons and 1, 2, 3, 4, 5, or 6 atoms independently selected from the group consisting of N, O, and S. In one embodiment, the hydrophobic moiety comprises 15-100 carbons and 1, 2, 3, 4, 5, or 6 atoms independently selected from the group consisting of N, O, and S. In one embodiment, the organic moiety R comprises 15-500 carbons and 1, 2, 3, 4, 5, or 6 atoms independently selected from the group consisting of N, O, and S. In one embodiment, the hydrophobic moiety has two components (FIG. 3), i.e., a linker, X, and an organic moiety, R.

The linker X can comprise CH₂, O, S, or NH with 1 to 100 unit of any combinations of CH₂, O, S and NH. In one embodiment, the linker X is a direct bond. In one embodiment, the linker X comprises 1-50 CH₂ groups and 1-10 groups selected from the group consisting of —C(═O)—. O, S, and NH. In one embodiment, the linker X comprises 1-50 CH₂ groups and 1, 2, 3, 4, 5, or 6 groups selected from the group consisting of —C(═O)—. O, S, and NH. In one embodiment, the linker X comprises 1-50 CH₂ groups and 1, 2, or 3 groups selected from the group consisting of —C(═O)—. O, S, and NH. In one embodiment, the linker X comprises 1-50 CH₂ groups and 1-10 groups selected from the group consisting of —C(═O)—. O, S, and NH. In one embodiment, the linker X comprises 1-50 CH₂ groups and 1, 2, 3, 4, 5, or 6 groups selected from the group consisting of —C(═O)—. O, S, and NH. In one embodiment, the linker X comprises 1-50 CH₂ groups and 1, 2, or 3 groups selected from the group consisting of —C(═O)—. O, S, and NH. In one embodiment, the linker X comprises 1-10 CH₂ groups and 1-5 groups selected from the group consisting of —C(═O)—. O, S, and NH. In one embodiment, the linker X comprises 1-10 CH₂ groups and 1, 2, or 3 groups selected from the group consisting of —C(═O)—. O, S, and NH. In one embodiment, the linker X comprises 1-10 CH₂ groups and 1 or 2 groups selected from the group consisting of —C(═O)—. O, S, and NH.

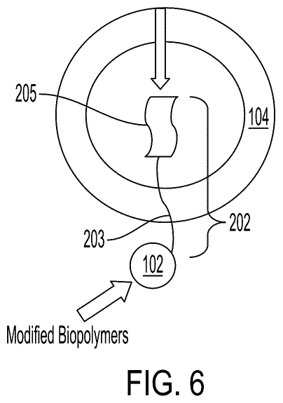

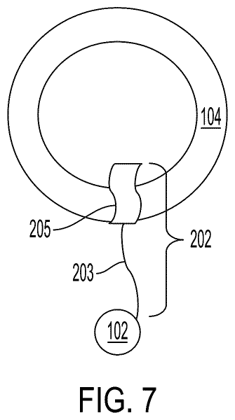

The organic moiety R can comprise various combinations of alkyl, alkenyl, alkynyl, aromatic rings, and heteroaromatic rings, with 1 to 100 of those units in any combinations. In one embodiment, the organic moiety (205, R) is selected so that it can be completely encapsulated (FIG. 6) in the single protein. In one embodiment, the organic moiety (205, R) is selected so that it can be partially encapsulated (FIG. 7) in a single protein to form a composition of the invention. In one embodiment, the organic moiety comprises a residue of a fatty acid. In one embodiment, the organic moiety comprises a residue of a fatty acid that comprises 8-50 carbons. In one embodiment, the organic moiety comprises a residue of a fatty acid that comprises 8-30 carbons. In one embodiment, organic moiety comprises a residue of a fatty acid that comprises 8-20 carbons. In one embodiment, the organic moiety comprises a residue of a fatty acid that comprises 15-50 carbons. In one embodiment, the organic moiety comprises a residue of a fatty acid that comprises 15-30 carbons. In one embodiment, the organic moiety comprises a residue of a fatty acid that comprises 15-20 carbons. In one embodiment, the organic moiety comprises 8-150 carbons. In one embodiment, the organic moiety comprises 8-50 carbons. In one embodiment, the organic moiety comprises 8-30 carbons. In one embodiment, the organic moiety comprises 15-150 carbons. In one embodiment, the organic moiety comprises 15-50 carbons. In one embodiment, the organic moiety comprises 15-30 carbons. In one embodiment, the organic moiety comprises 8-150 carbons and 1, 2, 3, 4, 5, or 6 atoms independently selected from the group consisting of N, O, and S. In one embodiment, the organic moiety comprises 8-50 carbons and 1, 2, 3, 4, 5, or 6 atoms independently selected from the group consisting of N, O, and S. In one embodiment, the organic moiety comprises 8-30 carbons and 1, 2, 3, 4, 5, or 6 atoms independently selected from the group consisting of N, O, and S. In one embodiment, the organic moiety comprises 15-150 carbons and 1, 2, 3, 4, 5, or 6 atoms independently selected from the group consisting of N, O, and S. In one embodiment, the organic moiety comprises 15-50 carbons and 1, 2, 3, 4, 5, or 6 atoms independently selected from the group consisting of N, O, and S. In one embodiment, the organic moiety comprises 15-30 carbons and 1, 2, 3, 4, 5, or 6 atoms independently selected from the group consisting of N, O, and S.

The incorporation of various units of hydrophobic moiety onto the charged biopolymers makes those biopolymers less hydrophilic, which can be easily to be encapsulated into the hydrophobic binding pockets of a single protein, such as albumins and antibodies. In addition, the “modified biopolymer” could reduce the enzymatic digestion of those biopolymers and could enhance the cell penetration of those biopolymers due to the protection of biopolymers by the “hydrophobic moieties”.

Biopolymers/: The term “biopolymer” includes biological-active polymers and oligomers that are used for clinical therapeutics and diagnostics. Three main categories of biopolymers are included in the invention, (a) Nucleic acids and derivatives, such as, natural and modified (base and backbone-modified) DNA, RNA, DNA/RNA, oligonucleotides, morpholino oligonucleotides, thio-substituted oligonucleotides, thiophosphoramidate morpholino oligonucleotides, and their derivatives that contains various fluorescent tags and metal complexes, etc; (b) peptides/proteins and their derivatives, such as natural and modified peptides, proteins and their derivatives that contains various fluorescent tags and metal complexes, etc; (c) Peptide nucleic acids (PNA) and derivatives, such as standard PNA and modified PNA and their derivatives that contains various fluorescent tags and metal complexes, etc.

SPE-Biopolymers or SPE-Modified Biopolymers prepared from the single protein encapsulation have many advantages over the unformulated biopolymers, (i): longer circulation lives because the encapsulation by a single protein prevents the enzymatic digestion of biopolymers/bio-oligomers; (ii) very water soluble because a single protein (albumin or antibody) can carry those biopolymers to be completely soluble in an aqueous media and no other toxic agents are needed, (iii) the targeted delivery, because albumins and antibodies prefer to enter the cancer cells, instead of the normal cells, also prefer to go the inflammation/infection sites; (iv) reduced toxicity because of the excellent water solubility and the targeted delivery.

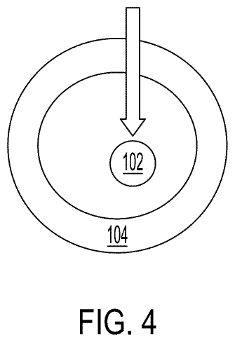

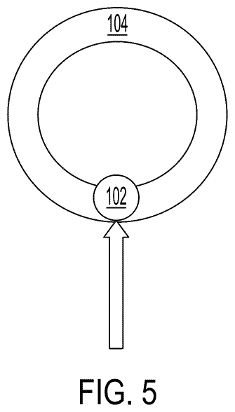

The term “tightly bound” means that all or a portion of the SPE-biopolymer or all or a portion of the SPE-modified biopolymer is encapsulated within the single protein; the SPE-biopolymers or SPE-modified biopolymers are not covalently bounded to the single protein either directly or through an intervening group. In one embodiment, the SPE-biopolymer is completely encapsulated by the single protein (FIG. 4). In another embodiment, only part of the surface area of the SPE-biopolymers is encapsulated by the single protein (FIG. 5). In another embodiment, one or more of the SPE-biopolymers may be completely encapsulated by the single protein and one or more the SPE-biopolymers may only have part of its surface area encapsulated by the single protein.

In one embodiment, the organic moiety 205 of the SPE-modified biopolymer is completely encapsulated by the single protein (FIG. 6). In another embodiment, only part of the surface area of the organic moiety 205 of the SPE-modified biopolymers is encapsulated by the single protein (FIG. 7). In another embodiment, one or more organic moiety of SPE-modified biopolymers may be completely encapsulated by the single protein and one or more organic moiety of the SPE-modified biopolymers may only have part of its surface area encapsulated by the single protein.

As used herein, the term “single protein” includes a single molecular species of a protein of both natural and synthetic origins, including proteins isolated from both living organisms and bioengineered systems. Furthermore, the protein may contain other non-protein components through either covalent linkage or noncovalent interaction. In one embodiment, the term does not include multimolecular species of a protein, such as a dimer, trimer, oligomer, or multimer. In one embodiment, the single protein is albumin, a globulin, a fibrinogen, IgA, IgM IgG, or another human protein.

As used herein, the term “albumin” includes any albumin. In one embodiment, the albumin is mammalian. In one embodiment, the albumin is human, cow, sheep, horse, or pig albumin. In one embodiment, albumin is non-mammalian. In one embodiment, the albumin is prepared from recombinant techniques. In the compositions of the invention, the albumin is not present in the form of particles, e.g. a nano-particle. Accordingly, the tightly-bound molecules of the pharmaceutical agent are encapsulated in pockets within the albumin structure, not within pores of an albumin nanoparticle.

As used herein, the term “globulin” includes any globulin. Globulins are a heterogeneous group of large serum proteins, not including albumin, which are soluble in salt solutions. There are three principal subsets of globulins, which are distinguished by their respective degrees of electrophoretic mobility: alpha globulin, beta globulin, and gamma globulin. Non-limiting examples of various globulins include clotting proteins, complement, many acute phase proteins, inrnunoglobulins (Igs), and lipoproteins. In one embodiment, the globulin is mammalian. In one embodiment, the globulin is human, cow, sheep, horse, or pig albumin. In one embodiment, globulin is non-mammalian. In one embodiment, the globulin is recombinant globulin. In one embodiment, the globulin is an immunoglobulin (Ig), such as an IgA, IgM, IgG, IgE or IgD antibody.

As used herein, the term “antibody” includes a single-chain variable fragment (scFv or “nanobody”), humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “fibrinogen” includes any fibrinogen. Fibrinogen is a soluble glycoprotein present in blood plasma, from which fibrin is produced by the action of the enzyme thrombin. In one embodiment, the fibrinogen is mammalian. In one embodiment, the fibrinogen is human, cow, sheep, horse, or pig fibrinogen. In one embodiment, fibrinogen is non-mammalian. In one embodiment, the fibrinogen is a recombinant fibrinogen.

As used herein, the term “polar organic solvent” includes solvents that are miscible with water or partially dissolved in water. For example, the term includes water miscible solvents or water partially dissolved solvents. The term “polar organic solvent” includes:
(1) Water soluble alcohols: methanol, ethanol, isopropanol, butanol, pentanol, t-butanols, etc
(2) Water soluble diols and triols, tetraols: ethylene glycol, propylene glycol, glycerol, etc
(3) Water soluble aldehydes and ketones: acetone, butanone, pentanones, hexanones, acetaldehyde, formyl aldehyde, propionaldehyde, butyraldehyde, etc
(4) Water soluble nitriles: acetonitrile, propionitrile, butanitrile, etc
(5) Water soluble polymers with low molecular weight: polyethylene glycols, polypropylene glycols, etc
(6) Water soluble amides: DMF, dimethylacetamide, dimethylpropanamide, etc,
(7) Water soluble ethers: diethyl ether, THF, dioxanes, etc
(8) All Other water soluble organic solvents: DMSO, etc

As used herein, the term “second biopolymer or modified biopolymer” includes “biopolymer or modified biopolymer”, as described previously. In one embodiment, the second biopolymer or modified biopolymer can be tightly bound within the single protein. In one embodiment, the “second biopolymer or modified biopolymer” contains NAs. In one embodiment, the “second biopolymer or modified biopolymer” contains peptides/proteins. In one embodiment, the “second biopolymer or modified biopolymer” contains PNAs. In one embodiment, the “second biopolymer or modified biopolymer” is an anticancer therapeutic, an antiinflammatory therapeutic, a CNS therapeutic, an antifungal therapeutic, or an antibiotic therapeutic, diagnostic probes. In one embodiment, the pharmaceutical agent is an anti-cancer compound.

HSA is well-known for its conformation changes when its environment is altered. It has been reported that HSA displayed different confirmations in acidic, neutral and basic conditions. HSA's conformation in a cosolvent, such as ethanol/water, or methanol/ethanol/water, or 1, 4-dioxane/water, or 2-butanone/ethanol/water, or acetone/water, or DMSO/water, or other organic solvents/water mixtures is dramatically different from the pure water (Borisover, M. D., et el., Thermochimica Acta, 1996, 284, 263-277). The literature shows that suspending HSA in the water/organic cosovents is accompanied by two main processes, (1) the water desorption-sorption, (2) the non-sorption that is attributed to rupture of protein-protein contact, depending on the nature of organic solvent and water content. Furthermore, the prepared HSA solution in the water/organic cosolvents results in the increase in the accessible surface areas, which has capacity to change the water sorption and calorific properties of the intended HSA suspension. HSA in the water/organic cosolvents is no longer in its natural state; it is partially denatured. Due to the fact that relative polarity of the cosolvent is lower than the pure water's, the resulting conformation changes of HSA in the desired organics/water mixture would allow some of the hydrophobic pockets to be opened up, allowing biopolymers or modified biopolymers to be tightly bound or encapsulated into these hydrophobic pockets., see FIG. 1 and FIG. 2. The compositions of the invention that have a biopolymer or modified biopolymer tightly bound within albumin as “SPE-Biopolymers or SPE-Modified Biopolymers” have novel properties, such as, for example, increased water solubility, reduced enzymatic digestions, targeted delivery for therapeutics and diagnostics.

In one embodiment of this invention, the single protein is dissolved in a cosolvents containing at least one water soluble organic solvent that helps the biopolymer or modified biopolymer to be able for being encapsulated into the single protein. The encapsulation process is monitored by UV or other instruments. Once the desired percent of single protein-encapsulated biopolymers or modified biopolymers is achieved, the encapsulation process is terminated and the final product, SPE-Biopolymers or Pre-Modified Biopolymers are prepared. After the filtration (e.g. through 0.22 um membrane or high speed centrifugation or other sterilization procedure), the concentrations of biopolymers or modified biopolymers can be quantified by UV spectrometer, HPLC or other methods after organic solvent extraction through the proteins precipitation. After quantification, the single protein-encapsulated biopolymers or modified biopolymers (SPE-Biopolymers or SPE-Modified Biopolymers” solutions can be frozen at −20° C. or lyophilized to powder products. In addition, in some embodiments, the single protein-encapsulated pharmaceuticals can be further purified via running through Sephadex G25 column, in which the large molecule, single protein-encapsulated biopolymers or modified biopolymers come out the first, followed by the un-capsulated biopolymers or modified biopolymers. In some embodiments, the single protein-encapsulated biopolymers or modified biopolymers can be further purified via dialysis using the dialysis pouch with different molecular weight cutoffs, in which the un-capsulated biopolymers or modified biopolymers with smaller molecular weights will be dialyzed out and the single protein-encapsulated biopolymers or modified biopolymers with macular weights >30 kd will be kept inside the dialysis bag. This invention provides novel method to prepare single protein-encapsulated biopolymers or modified biopolymers without chemically modifying structures of the single protein.

HSA is a biopolymer, with a molecular weight at about 66,000 g/mole with a particle size at about 10 nm, determined by dynamic light scattering (DLS). In the compositions of the invention the particle size of the albumin having one or more biopolymer or modified biopolymer encapsulated therein does not change. In one embodiment the albumin having one or more biopolymer or modified biopolymer tightly bound therein is soluble in water. In another embodiment the albumin having one or more biopolymer or modified biopolymer encapsulated therein is soluble in water at a pH in the range of from about 6 to about 8.

If the biopolymer or modified biopolymer is an anticancer therapeutic, the composition comprising a single protein having one or more biopolymer or modified biopolymer encapsulated therein may increase the MTD of the biopolymers, because the encapsulated biopolymers or modified biopolymers prefer to go to the cancer cells and will have fewer interactions with the normal cells. If the biopolymer or modified biopolymer is an antibiotic or antifungal therapeutics, the composition comprising the single protein having one or more biopolymer or modified biopolymer tightly bound therein may decrease the MIC (minimum inhibitory concentrations) against the microorganism, because the tightly-bound “SPE-Biopolymers or SPE-Modified Biopolymers” favorably bind to the surface of both bacteria (gram-positive and gram-negative) and fungi. Therefore, in some embodiments the single protein-tightly bound biopolymers or modified biopolymers can be characterized by the comparison of their MTD or MIC to that of free form of biopolymers.

As described in the previous section, during the preparation process, the intended biopolymers or modified biopolymers molecules are entrapped in the binding pockets of the single protein once the single protein-tightly bound biopolymers or modified biopolymers are successfully prepared. Compared to the free form, the encapsulated biopolymers or modified biopolymers are surrounded by different environments, which could cause the changes of their UV spectra or florescence emission spectroscopy. The carrier, binding and proximity relationships of the encapsulated biopolymers or modified biopolymers molecules can be characterized and analyzed using absorption, fluorescence, FTIR, or circular dichroism.

In one embodiment, the invention provides a composition comprising, a single protein having one or more biopolymers or modified biopolymers tightly bound therein, wherein the single protein having one or more biopolymers or modified biopolymers tightly bound therein has a fluorescence emission that differs from the single protein alone. In one embodiment, the invention provides a composition comprising, a single protein having one or more biopolymers or modified biopolymers tightly bound therein, wherein the single protein having one or more biopolymers or modified biopolymers tightly bound therein has a fluorescence emission that is greater than the fluorescence emission of the single protein alone. In one embodiment, the invention provides a composition comprising, a single protein having one or more biopolymers or modified biopolymers tightly bound therein, wherein the single protein having one or more biopolymers or modified biopolymers tightly bound therein has a fluorescence emission that is less than the fluorescence emission of the single protein alone.

In one embodiment, the invention provides a composition comprising, a single protein having one or more biopolymers or modified biopolymers tightly bound therein, wherein the composition has a higher therapeutic efficacy in vivo than the biopolymer or modified biopolymer alone. In one embodiment, the invention provides a composition comprising, a single protein having one or more biopolymers or modified biopolymers tightly bound therein, wherein the composition has a therapeutic efficacy in vivo that is at least 2 times the therapeutic efficacy of the biopolymer or modified biopolymer alone. In one embodiment, the invention provides a composition comprising, a single protein having one or more biopolymers or modified biopolymers tightly bound therein, wherein the composition has a therapeutic efficacy in vivo that is at least 5 times the therapeutic efficacy of the biopolymer or modified biopolymer alone.

In one embodiment, the invention provides a composition comprising, a single protein having one or more biopolymers or modified biopolymers tightly bound therein, wherein the composition is more soluble in aqueous media (e.g., a solution comprising water or water) than the biopolymer or modified biopolymer alone. In one embodiment, the invention provides a composition comprising, a single protein having one or more biopolymers or modified biopolymers tightly bound therein, wherein the composition has a water solubility that is at least 2 times more than the water solubility of the biopolymer or modified biopolymer alone. In one embodiment, the invention provides a composition comprising, a single protein having one or more biopolymers or modified biopolymers tightly bound therein, wherein the composition has a water solubility that is at least 5 times the water solubility of the biopolymer or modified biopolymer alone.

The formulations of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the formulations may be combined with one or more excipients and used in the form of ingestible tablets, buccal tablets, troches, capsules, elixirs, suspensions, syrups, wafers, and the like. Such compositions and preparations should contain at least 0.1% of the formulation. The percentage of the compositions and preparations may, of course, be varied and may conveniently be between about 2 to about 60% of the weight of a given unit dosage form.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the formulations may be incorporated into sustained-release preparations and devices.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the formulations in the required amount in the appropriate solvent with variety of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the formulations can be determined by comparing their in vitro activity, and in vivo activity in animal models. Methods for the extrapolation of effective dosages in mice, and other animals, to humans are known to the art; for example, see U.S. Pat. No. 4,938,949.

The amount of the formulation required for use in treatment will vary with the particular formulation selected, with the route of administration, with the nature of the condition being treated and with the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Formulations of the invention can also be administered in combination with other therapeutic agents. Accordingly, in one embodiment the invention also provides a composition comprising a formulation of the invention, at least one other therapeutic agent, and a pharmaceutically acceptable diluent or carrier. The invention also provides a kit comprising a formulation of the invention, at least one other therapeutic agent, packaging material, and instructions for administering the formulation and the other therapeutic agent or agents to an animal.

The unusually long half-life of HSA or IgG is maintained mainly by FcRn or the Brambell receptor (Brambell, F. W., et el., Nature, 1964, 203, 1352-1354), which is expressed in a wide variety of tissues and organs (Sockolosky, J. T., and Szoka, F. C., Adv Drug Deliv Rev, 2015, 91, 109-124). This major histocompatibility complex class I-related receptor was originally discovered to play an important role in the delivery of IgGs from the mother to the young, regulate serum IgG concentration, and maintain the long half-life of IgGs in the serum. It was later discovered that FcRn can bind both IgG and HSA at different sites and is responsible for the long half-lives of both IgGs and HAS (Chaudhury, C., J Exp Med, 2003, 197, 315-322; Anderson, C. L., et el., Trends Immunol, 2006, 27, 343-348; Chaudhury, C., et el., Biochemistry, 2006, 45, 4983-4990; and Kim, J., Bronson, et el., Am J Physiol Gastrointest Liver Physiol, 2006, 290, G352-360). As expected, FcRn mutation was found to cause familial hypercatabolic hypoproteinemia (Wani, M. A., et el., Proc Natl Acad Sci USA, 2006, 103, 5084-5089).

The mechanism of HSA rescue and recycling has been elucidated and involves:
1. FcRn binding HSA in the endosome due to high affinity at acidic pH,
2. The resulting FcRn-HSA complex (1:1 ratio) is sent back to the bloodstream,
3. The FcRn-HSA complex dissociates due to low affinity at pH 7.4, releasing HSA back in the circulation.
   In cells that express low levels of FcRn, HSA would be endocytosed to lysosomes, where it is degraded to amino acids. As a result, this FcRn-mediated recycling pathway is a major factor contributing to the long half-life of both IgGs and HSA in human, and has significant pathophysiological and therapeutic implications.

From publicly available protein expression databases (Uhlen, M., et el., Science, 2015, 347, 1260419; Lindskog, C., Expert Rev Proteomics, 2016, 13, 627-629; Tang, Z., Nucleic Acids Res, 2017, 45, W98-W102; Uhlen, M., Science, 2017, 357; and Papatheodorou, I., et el., Nucleic Acids Res, 2018, 46, D246-D251), differences in FcRn expression between normal and cancer tissues from different human organs was investigated. A FcRn expression comparison was made in 31 different tissues. Among them, more than half ( 16/31) of cancer tissues express less FcRn than their normal tissue counterparts, while about more than a quarter ( 12/31) of cancer tissues express more FcRn, with only 3 having similar FcRn levels. The first group (16 cancers, table 1) is the specific targets for SPE-anticancer drugs.

TABLE 1
Tumors Targeted for treatment (ratio of FcRn in normal/tumor tissue ≥ 1.19 )

| Tumor | | Ratio of FcRn in |
|---|---|---|
| Abbreviation | Tumor Type | Normal/Tumor tissue |
| ACC | Adrenocortical carcinoma | 2.63 |
| BLCA | Bladder Urothelial Carcinoma | 1.81 |
| BRCA | Breast invasive carcinoma | 1.66 |
| CESC | Cervical squamous cell carcinoma and | 3.48 |
| | endocervical adenocarcinoma | |
| CHOL | Cholangiocarcinoma | 1.75 |
| DLBC | Lymphoid Neoplasm Diffuse Large B- | 1.55 |
| | Lymphoma | |
| KICH | Kidney Chromophobe | 1.92 |
| LUAD | Lung adenocarcinoma | 1.62 |
| LUSC | Lung squamous cell carcinoma | 3.14 |
| OV | Ovarian serous cystadenocarcinoma | 2.30 |
| PCPG | Pheochromocytoma and Paraganglioma | 1.33 |
| PRAD | Prostate adenocarcinoma | 1.64 |
| SARC | Sarcoma | 1.19 |
| THCA | Thyroid carcinoma | 1.39 |
| UCEC | Uterine Corpus Endometrial Carcinoma | 1.61 |
| UCS | Uterine Carcinosarcoma | 1.42 |

FIG. 1 illustrates an encapsulation process 100 combining a biopolymer 102 and a single protein 104 to form a composition of the invention 106.

FIG. 2 shows an illustration 200 of the relationship between biopolymers 102, single proteins 104, hydrophobic moieties 202, modified biopolymers 206, and an encapsulation process for combining a modified biopolymer 206 and a single protein 104 to form a structure 204 including a single protein 104 and a modified biopolymer 206 tightly bound therein.

FIG. 4 illustrates a biopolymer 102 completely encapsulated by a single protein 104.

FIG. 5 illustrates a biopolymer 102 wherein only part of the surface area of the biopolymer is encapsulated by a single protein 104.

FIG. 6 illustrates a biopolymer 102 that is linked to a hydrophobic moiety 202 that comprises linker 203 and a organic group 205, where the organic group 205 is completely encapsulated by a single protein 104.

FIG. 7 illustrates a biopolymer 102 that is linked to a hydrophobic moiety 202 that comprises linker 203 and an organic group 205, where the organic group 205 is partially encapsulated in the single protein 104.

The invention will now be illustrated by the following non-limiting Examples.

### EXAMPLES

### Example 1: Single Protein Encapsulation of PNA 12065J to Form SPEPNA12065J

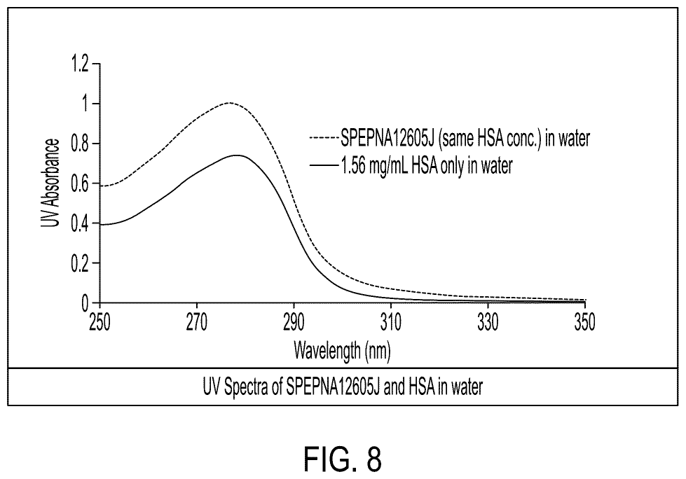

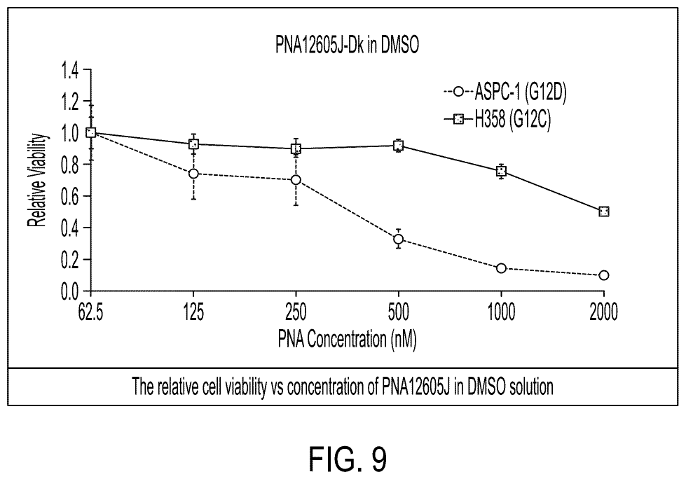

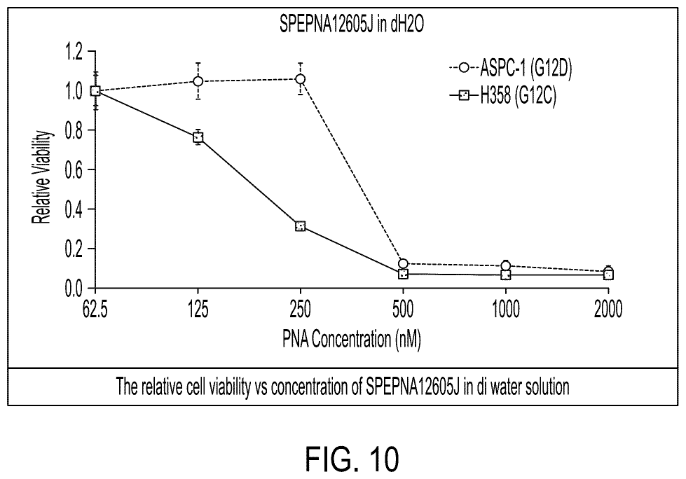

PNA12605J is a water insoluble PNA with the following sequence, HPLC chromatogram and MALDI MS (Table 2).
(1) Preparation of PNA12605J methanol solution: to 1.5 mg of PNA12605J powder was added 1.0 mL of methanol, shaking well, followed by adding 1.0 mL of DI water. The mixture was sonicated for 15 min, a clear methanol/water solution of PNA12605J was prepared with a concentration of [PNA12605J]=0.75 mg/mL.
(2) Encapsulation of PNA12605J: To 100 mg of HSA powder was added 10 mL of DI water, after stirring for 10 min, the HSA solution was prepared. Into this HSA solution was added 3.2 mL of 50% ethanol/water (v/v), and 350 uL of 1.0 M NaOH solution. The mixture was stirred in a 37° C.—oil bath under N₂ atmosphere. After stirring for 30 min, 0.5 mL of the above PNA12605J solution was added by a pipette. After stirring for 1 h, another 0.5 mL of the above PNA12605J solution was added by a pipette. After stirring for 1 h, another 0.5 mL of the above PNA12605J solution was added by a pipette. After stirring for 1 h, another 0.5 mL of the above PNA12605J solution was added by a pipette. After the addition of PNA12605 J methanol solution was completed, the mixture was stirred at 37° C. overnight. After cooling to room temperature, the pH of the solution was adjusted to 7.4 by adding 0.1 M HCl solution. The mixture was concentrated to 6 mL by the high vacuum pump and the mixture was centrifuged at 12000 RMP for 5 min, the top solution was transferred into several 15 mL of centrifuge tubes and lyophilized into the powered product. Each tube of SPEPNA12605J contained 0.31 mg of PNA12605J (equivalent) and 21.7 mg of HSA. Into a tube containing SPEPNA12605J was added 1.0 mL of DI water, the clear solution was prepared. UV spectra of SPEPNA12605J solution was measured and compared to the UV spectra of the same concentration of HSA, shown in FIG. 8. It is clear that PNA12605J was encapsulated to HSA to form a water soluble SPEPNA12605J.
(3) In vitro evaluation of SPEPNA12605J and PNA12065J on anticancer activities: Two cancer cell lines, ASPC-1 (pancreatic cancer, KRAS mutation at G12D), NCI-H358 (non-small cell lung cancer, KRAS mutation at G12C) were used for in vitro evaluation of PNA12605J and SPEPNA12605J for their anticancer activity using the standard cell culture procedures. The relative cell viability vs the concentrations of PNA were shown in FIG. 9 and FIG. 10. IC50 and IC90 for PNA12605J and SPEPNA12605J, obtained from FIG. 9 and FIG. 10, are summarized in Table 3. Those data demonstrated that the encapsulation of PNA12605J by HSA, not only made PNA12605J water soluble (NO need of toxic DMSO for IV injection), but also substantially enhanced PNA12605J's antitumor efficacy.

TABLE 3
Summary of In vitro evaluation results on both cancer cells

| | ASPC-1 | | NCI-H358 | |
|---|---|---|---|---|
| | IC50 (nM) | IC90 (nM) | IC50 (nM) | IC90 (nM) |
| PNA12605J | 350 | 1000 | 2000 | >2000 |
| SPEPNA12605J | 350 | 500 | 200 | 500 |

### Example 2: Single Protein Encapsulation of PNA 118C to Form SPEPNA118C

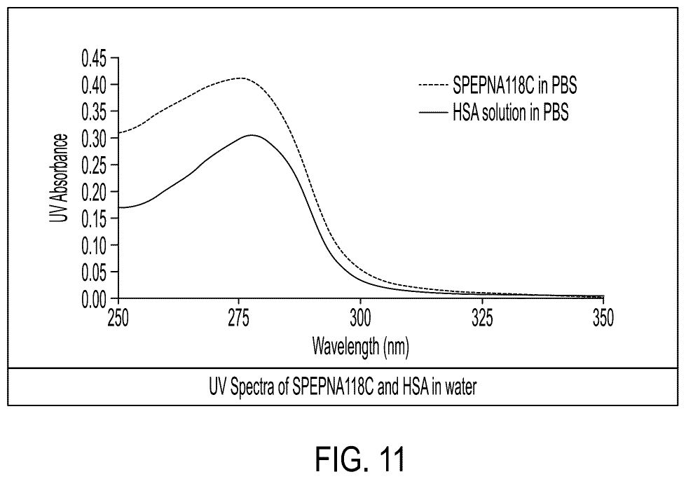

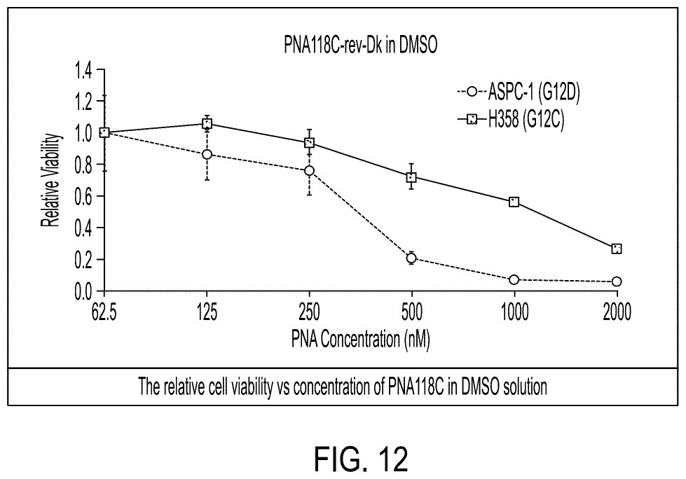

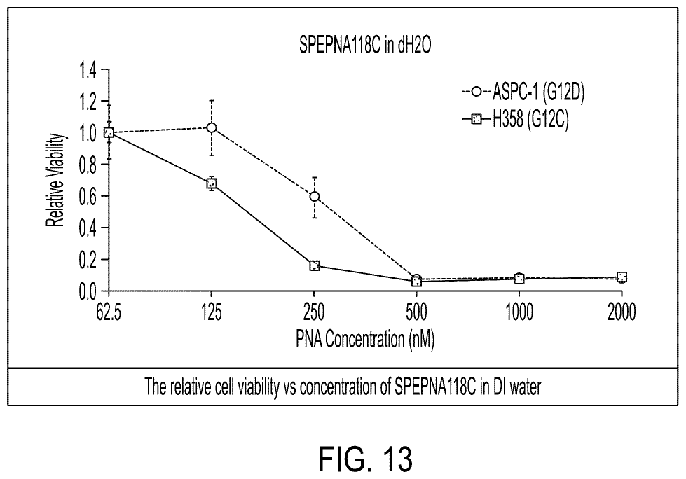

PNA118C is a water insoluble PNA with the following sequence, HPLC chromatogram and MALDI MS (Table 4).
(1) Preparation of PNA11C methanol solution: to 1.5 mg of PNA118C powder was added 1.0 mL of methanol, shaking well, followed by adding 1.0 mL of DI water. The mixture was sonicated for 15 min, a clear methanol/water solution of PNA118C was prepared with a concentration of [PNA118C]=0.75 mg/mL.
(2) Encapsulation of PNA118C: To 100 mg of HSA powder was added 10 mL of DI water, after stirring for 10 min, the HSA solution was prepared. Into this HAS solution was added 3.2 mL
of 50% ethanol/water (v/v), and 350 uL of 1.0 M NaOH solution. The mixture was stirred in a 37° C.—oil bath under N₂ atmosphere. After 30 min, 0.5 mL of the above PNA118C solution was added by a pipette. After stirring for 1 h, another 0.5 mL of the above PNA118C solution was added by a pipette. After stirring for 1 h, another 0.5 mL of the above PNA118C solution was added by a pipette. After stirring for 1 h, another 0.5 mL of the above PNA118C solution was added by a pipette. After the addition of PNA118C methanol/water solution was completed, the mixture was stirred at 37° C. overnight. After cooling to room temperature, the pH of the solution was adjusted to 7.4 by adding 0.1 M HCl solution. The mixture was concentrated to 7.35 mL and the mixture was centrifuged at 12000 RMP for 4 min, the top solution was transferred into several 15 mL of centrifuge tubes and lyophilized into the powered product. Each tube of SPEPNA118C contained 0.34 mg of PNA118C (equivalent) and 27 mg of HSA. Into a tube containing SPEPNA118C was added 1.0 mL of PBS buffer, the clear solution was prepared. UV spectra of SPEPNA118C solution was measured and compared to the UV spectra of the same concentration of HSA in PBS, shown in FIG. 11. It is clear that PNA118C was encapsulated to HSA to form a water soluble SPEPNA118C.
(3) In vitro evaluation of SPEPNA118C and PNA118C on anticancer activities: Two cancer cell lines, ASPC-1 (pancreatic cancer, KRAS mutation at G12D), NCI-H358 (non-small cell lung cancer, KRAS mutation at G12C) were used for in vitro evaluation of PNA118C and SPEPNA118C for their anticancer activity using the standard cell culture procedures. The relative cell viability vs the concentrations of PNA were shown in FIG. 12 and FIG. 13. IC50 and IC90 for PNA118C and SPEPNA118C, obtained from FIG. 12 and FIG. 13, are summarized in Table 5. Those data demonstrated that the encapsulation of PNA118C by HSA, not only made PNA118C water soluble (No need of toxic DMSO for IV injection), but also substantially enhanced PNA118's antitumor efficacy.

TABLE 5
Summary of In vitro evaluation results on both cancer cells

| | ASPC-1 | | NCI-H358 | |
|---|---|---|---|---|
| | IC50 (nM) | IC90 (nM) | IC50 (nM) | IC90 (nM) |
| PNA118C | 350 | 1000 | 1200 | >2000 |
| SPEPNA118C | 300 | 500 | 200 | 500 |

What is claimed is:

1. A composition comprising, a single protein having one or more biopolymers or modified biopolymers tightly bound therein.

2. The composition of claim 1, wherein each biopolymer or modified biopolymer comprises a nucleic acid.

3. The composition of claim 1, wherein each biopolymer or modified biopolymer comprises a peptide or protein.

4. The composition of claim 1, wherein each biopolymer or modified biopolymer comprises a peptide nucleic acid.

5. The composition of claim 1, wherein the single protein is an albumin.

6. The composition of claim 1, wherein the single protein is a globulin.

7. The composition of claim 1, wherein the single protein is a fibrinogen.

8. The composition of claim 1, wherein the single protein is IgA, IgM, or IgG.

9. The composition of claim 1, wherein a plurality (e.g., at least 2, 3, 4, 5, or 6) of biopolymers or modified biopolymers are tightly bound within each single protein.

10. The composition of claim 1, wherein at least one biopolymer or modified biopolymer is tightly bound within each single protein.

11. The composition of claim 1, wherein one or more biopolymer or modified biopolymer is completely encapsulated within the single protein.

12. The composition of claim 1, wherein only part of the surface area of one or more biopolymer or modified biopolymer is encapsulated by the single protein.

13. The composition of claim 1, which further comprises water and one or more water soluble organic solvents.

14. The composition of claim 2, wherein nucleic acid is selected from the group consisting of natural and modified (base and backbone-modified) DNA, RNA, DNA/RNA, oligonucleotides, morpholino oligonucleotides, thio-substituted oligonucleotides, thiophosphoramidate morpholino and oligonucleotides, that optionally comprise a fluorescent tag or a metal complex.

15. The composition of claim 3, wherein the peptide or protein is a natural or modified peptide or protein, that optionally comprises a fluorescent tag or a metal complex.

16. The composition of claim 4, wherein the peptide nucleic acid (PNA) is a standard PNA or a modified PNA that optionally comprises a fluorescent tag or a metal complex.

17. A composition comprising, a single protein having one or more biopolymers tightly bound therein.

18. A composition comprising, a single protein having one or more modified biopolymers tightly bound therein.

19. The composition of claim 17, wherein a plurality of biopolymers are tightly bound within each single protein.

20. The composition of claim 18, wherein a plurality of modified biopolymers are tightly bound within each single protein.

21. The composition of claim 17, wherein one or more biopolymers is completely encapsulated within the single protein.

22. The composition of claim 18, wherein one or more modified biopolymers is completely encapsulated within the single protein.

23. The composition of claim 18, wherein the one or more modified biopolymers each comprises a charged backbone and is linked to a hydrophobic moiety, which hydrophobic moiety is completely encapsulated within the single protein.

24. The composition of claim 18, wherein the one or more modified biopolymers each comprises a charged backbone and is linked to a hydrophobic moiety, which hydrophobic moiety is partially encapsulated within the single protein.

25. The composition of claim 18, wherein the one or more modified biopolymers are not charged and are linked to a hydrophobic moiety, which hydrophobic moiety is completely encapsulated within the single protein.

26. The composition of claim 18, wherein the one or more modified biopolymers are not charged and are linked to a hydrophobic moiety, which hydrophobic moiety is partially encapsulated within the single protein.
